(19) United States
(12) Patent Application Publication
(10) Pub. No.: US 2012/0071671 A1
(43) Pub. Date: Mar. 22, 2012

(54) CATALYST AND METHOD FOR PARTIALLY OXIDIZING HYDROCARBONS

(75) Inventors: Andrey Karpov, Mannheim (DE); Hartmut Hibst, Schriesheim (DE); Christine Deißler, Mannheim (DE); Cornelia Katharina Dobner, Ludwigshafen (DE); Frank Rosowski, Mannheim (DE); Nadine Brem, Muhlhausen-Rettigheim (DE); Stephan Schunk, Heidelberg-Rohrbach (DE)

(73) Assignee: BASF SE, Ludeigshafende (DE)

(21) Appl. No.: 13/375,019

(22) PCT Filed: May 28, 2010
(86) PCT No.: PCT/EP2010/057375

§ 371 Date: Nov. 29, 2011

(30) Foreign Application Priority Data
May 29, 2009 (EP) 09161538.5

(51) Int. Cl. C07D 307/89; B01J 27/199; B01J 23/68

(52) U.S. Cl. 549/248; 502/312; 502/209

(57) ABSTRACT

The invention relates to a catalyst for partially oxidizing hydrocarbons in the gas phase, containing a multi-metal oxide of the general formula (I), AgaMObVcMdOe.f H2O (I), wherein M stands for at least one element selected from among Li, Na, K, Rb, Cs, Be, Mg, Ca, Sr, Ba, B, Al, Ga, In, Si, Sn, Pb, P, Sb, Bi, Y, Ti, Zr, Hf, V, Nb, Ta, Cr, W, Mn, Re, Fe, Ru, Os, Co, Rh, Ir, Ni, Pd, Pt, Cu, Au, Zn, Cd, La, Ce, Pr, Nd, Sm, Eu, Gd, Tb, Dy, Ho, Er, Tm, Yb, Lu, and U, a has a value of 0.5 to 1.5, b has a value of 0.5 to 1.5, c has a value of 0.5 to 1.5, a+b+c has the value 3, d has a value of less than 1, e means a number that is determined by the valence and frequency of the elements other than oxygen in the formula (I), f has a value of 0 to 20, which multi-metal oxide exists in a crystal structure, the X-ray powder diffractogram of which is characterized by diffraction reflections at a minimum of 5 lattice distances selected from among d=4.53, 3.38, 3.32, 3.23, 2.88, 2.57, 2.39, 2.26, 1.83, 1.77 AA (+−0.04 AA).

The invention relates to a catalyst for partial oxidation of hydrocarbons in the gas phase, especially for partial oxidation of alkylaromatics to aromatic alcohols, aldehydes, carboxylic acids and/or carboxylic anhydrides, and to a process using the catalyst.

In contrast to the total oxidation to the carbon oxides CO and CO₂, the partial oxidation of hydrocarbons is understood to mean the oxidation of the hydrocarbons to unsaturated compounds and/or oxygenates. Examples of industrial importance relate, for example, to the partial oxidation of butane to maleic anhydride, of propane or propene to acrolein or acrylic acid, or of alkylaromatics to aromatic carboxylic acids and/or carboxylic anhydrides such as phthalic anhydride. In catalytic gas phase oxidation, a mixture of an oxygenous gas, for example air, and the hydrocarbon to be oxidized is passed at elevated temperature through a bed of a catalyst.

This partial oxidation probably proceeds by a combined parallel and subsequent step mechanism. The reactant is oxidized at the catalyst surface consecutively via intermediate oxidation products up to the end product. At each stage, the intermediate oxidation product can either be oxidized further or desorbed from the catalytically active surface. Total oxidation proceeds in a competing parallel reaction, proceeding either directly from the reactant or from an intermediate of the selective route. The selective oxidation of a hydrocarbon to a product of value forms many additional reaction products. These can be divided essentially into two subgroups. One group has a lower ratio of C/O atom numbers compared to the product of value. These underoxidation products can be converted to the target product. The second group includes the overoxidation products and the carbon oxides CO and CO₂ (often combined as COₓ).

Even small enhancements in yield of the industrial preparation processes lead to significant economic advantages. It is desirable to possess catalysts which lead with high selectivity to the desired product of value and/or underoxidation products thereof. The underoxidation products can be oxidized further to the desired product of value by known processes.

DE 198 51 786 describes a multimetal oxide comprising silver oxide and vanadium oxide. Catalysts prepared therefrom find use for the partial oxidation of aromatic hydrocarbons.

EP-A 756 894 describes multimetal oxide materials which comprise an active phase and a promoter phase. The phases are present relative to one another as in a mixture of finely divided active and promoter phases. The active phase comprises molybdenum, vanadium and at least one of the elements tungsten, niobium, tantalum, chromium and cerium; the promoter phase comprises copper and at least one of the elements molybdenum, tungsten, vanadium, niobium and tantalum. The multimetal oxide materials are used, for example, as catalysts for the oxidation of acrolein to acrylic acid.

NL 720 99 21 discloses a process for continuously preparing benzaldehyde by oxidation of toluene in the gas phase in the presence of a catalyst which comprises molybdenum and at least one further element M selected from nickel, cobalt, antimony, bismuth, vanadium, phosphorus, samarium, tantalum, tin and chromium, in an atomic M/Mo ratio of less than 1:1.

EP-A 0 459 729 describes a catalyst for the preparation of substituted benzaldehydes, whose catalytically active material consists of an oxide of the formula VₐMo_{b}X_{c}Y_{d}Oₑ in which X is Na, K, Rb, Cs or Th, and Y is Nb, Ta, P, Sb, Bi, Te, Sn, Pb, B, Cu or Ag.

E. Wenda and A. Bielański examine, in J. Thermal Analysis and calorimetry, Vol. 92 (2008) 3, 931-937 and Polish J. Chem., 82, 1705-1709 (2008), the phase diagram of the V₂O₅—MoO₃—Ag₂O system. The authors describe the occurrence of the ternary phase AgVMoO₆.

C. R. Acad. Sc. Paris, t. 264 (1967), Series C, 1477-1480 and Bull. Soc. fr. Mineral. Cristallogr. (1968), 91, 325-331 address a crystallographic phase of the composition AgₓVₓMo₁₋ₓO₃ (where 0.44×0.50).

It is an object of the invention to discover novel multimetal oxides as catalysts for the partial oxidation of hydrocarbons, which lead to the desired products of value with high selectivity, especially for the partial oxidation of alkylaromatics to aromatic alcohols, aldehydes, carboxylic acids and/or carboxylic anhydrides.

The object is achieved by a catalyst for the partial oxidation of hydrocarbons in the gas phase, which comprises a multimetal oxide which consists essentially of a compound of the general formula (I)

AgₐMo_{b}V_{c}M_{d}Oₑ*f H₂O (I)

in which
M is at least one element selected from Li, Na, K, Rb, Cs, Be, Mg, Ca, Sr, Ba, B, Al, Ga, In, Si, Sn, Pb, P, Sb, Bi, Y, Ti, Zr, Hf, V, Nb, Ta, Cr, W, Mn, Re, Fe, Ru, Os, Co, Rh, Ir, Ni, Pd, Pt, Cu, Au, Zn, Cd, La, Ce, Pr, Nd, Sm, Eu, Gd, Tb, Dy, Ho, Er, Tm, Yb, Lu, U,
a has a value of 0.5 to 1.5,
b has a value of 0.5 to 1.5,
c has a value of 0.5 to 1.5,
a+b+c has the value of 3,
d has a value of less than 1,
e is a number which is determined by the valency and frequency of the elements in the formula I other than oxygen,
f has a value of 0 to 20,
which is present in a crystal structure whose powder x-ray diffractogram is characterized by reflections at at least 5, preferably at least 7, especially all, interplanar spacings selected from d=4.53, 3.38, 3.32, 3.23, 2.88, 2.57, 2.39, 2.26, 1.83, 1.77 Å (±0.04 Å).

The invention also relates to a process for partial oxidation of hydrocarbons, in which a gaseous stream comprising at least one hydrocarbon and molecular oxygen is passed over a bed of the catalyst.

The inventive catalysts are based on a ternary oxide of silver, molybdenum and vanadium. Incorporation of M atoms into the structure allows the catalytic properties of the multimetal oxide with regard to its activity and selectivity to be modified.

In the formula I, a preferably has a value of 0.7 to 1.3, especially of 0.8 to 1.2.

In the formula I b preferably has a value of 0.7 to 1.3, especially of 0.8 to 1.2.

In the formula I c preferably has a value of 0.7 to 1.3, especially of 0.8 to 1.2.

In embodiments of the gas phase oxidation catalyst, M is at least one element selected from Cs, B, Al, Ga, Pb, P, Sb, Bi, Nb, Cr, W, Re, Fe, Co, Cu, Pt, Pd, Zn, La, Ce, especially at least one element selected from P, Ce, Sb, Bi, Cs, Nb, W, B, Cu, Fe. In the formula I d has, for example, a value of 0 to 0.5, for example of 0.001 to 0.2.

In other embodiments, d has the value 0, i.e. an element M is absent.

In this application, the x-ray reflections are reported in the form of the interplanar spacings d [Å] which are independent of the wavelength of the x-radiation used, and which can be calculated from the diffraction angle measured by means of the Bragg equation.

In general, the complete powder x-ray diffractogram of the multimetal oxide of the formula I has reflections including the 11 listed in table 1. Less intense reflections of the powder x-ray diagram of the multimetal oxides of the formula I have not been included in table 1.

TABLE 1

| Reflection | d [Å] |
|---|---|
| 1 | 4.53 ± 0.06 |
| 2 | 3.38 ± 0.04 |
| 3 | 3.32 ± 0.04 |
| 4 | 3.23 ± 0.04 |
| 5 | 2.88 ± 0.04 |
| 6 | 2.57 ± 0.04 |
| 7 | 2.39 ± 0.04 |
| 8 | 2.26 ± 0.04 |
| 9 | 1.83 ± 0.04 |
| 10 | 1.77 ± 0.04 |
| 11 | 2.48 ± 0.04 |

The multimetal oxide is obtainable in various ways. It is obtained, for example, by reaction of at least one silver source, of at least one molybdenum source, of at least one vanadium source and optionally of a source of the element M. In general, it is followed by a thermal treatment at a temperature of at least 200° C.

In general, a silver source, a molybdenum source, a vanadium source and optionally a source of the element M are mixed intimately with one another. The mixing can be effected in dry form, but is preferably effected in wet form, for example in a solution and/or suspension in a solvent. The solvents used may be polar organic solvents, such as alcohols, polyols, polyethers or amines, e.g. pyridine, preference being given to using water as the solvent.

The silver sources, molybdenum sources, vanadium sources and sources of the element M used are the elements themselves, or oxides or compounds of the elements which are convertible to oxides in the course of heating, at least when heated in the presence of oxygen. These include hydroxides, oxide hydroxides, polyoxometallates, carboxylates, carbonates and especially nitrates.

Suitable silver sources are, for example, silver powder, silver oxides (for example Ag₂O), silver nitrate or silver acetate. Preference is given to using silver nitrate or silver acetate.

Suitable molybdenum sources are, for example, molybdenum powder, ammonium molybdate or ammonium polymolybdates (e.g. ammonium dimolybdate, ammonium heptamolybdate, ammonium octamolybdate, ammonium decamolybdate), molybdenum oxides such as MoO₃, MoO₂, molybdenum halides, molybdenum oxyhalides and molybdenum organyls. Owing to its general availability and good solubility, preference is given to using ammonium heptamolybdate.

Suitable vanadium sources are, for example, vanadium powder, ammonium monovanadate, ammonium polyvanadates (e.g. ammonium divanadate), ammonium metavanadate, vanadium oxides (such as V₂O₅, VO₂, V₂O₃ or VO), vanadium halides, vanadium oxyhalides and vanadium organyls. Alternative vanadium sources are sodium ammonium vanadate, potassium metavanadate and potassium orthovanadate.

Owing to its general availability and good solubility, preference is given to using ammonium metavanadate.

The sources of the element M selected are generally those compounds which are soluble in the solvent used. It is possible, for example, to use the carboxylates, especially the acetates or oxalates, nitrates, oxides, carbonates or halides. Element- oxygen acids or the ammonium salts thereof can be used when M is, for example, P. Formulations composed of nanoparticles of oxides or hydroxides of the elements M can likewise be used. In addition, polyanions such as heteropolyacids of the Anderson, Dawson or Keggin type or non-Keggin type can be used as sources for the elements M.

According to the desired chemical composition of the multimetal oxide of the formula (I), it is prepared by mixing amounts, which are evident from a, b, c and d of the formula (I), of silver source, molybdenum source, vanadium source and source of the element M.

The mixing of the silver source, molybdenum source, vanadium source and source of the element M can generally be carried out at room temperature or at elevated temperature. In general, the reaction is undertaken at temperatures of 20 to 375° C., preferably at 20 to 100° C. and more preferably at 60 to 100° C. When the reaction temperature is above the boiling point temperature of the solvent used, the reaction is appropriately performed under the autogenous pressure of the reaction system in a pressure vessel. Preference is given to selecting the reaction conditions such that the reaction can be carried out at atmospheric pressure. The duration of this reaction may, depending on the type of the starting materials used and the thermal conditions employed, be a few minutes to several days.

The mixture thus formed can be isolated from the reaction mixture and be stored until further use. The isolation can be effected, for example, by removing the solvent and drying the resulting solid. Suitable apparatus for drying includes customary driers, such as roll driers or freeze driers. Particularly advantageously, the drying of the resulting solution and/or suspension is carried out by means of spray-drying. The spray-drying is generally undertaken under atmospheric pressure or reduced pressure. The entrance temperature of the drying gas used is determined by the pressure employed and solvent used. In general, the drying gas used is air, but it is of course also possible to use other drying gases such as nitrogen or argon. The entrance temperature of the drying gas into the spray drier is advantageously selected such that the exit temperature of the drying gas cooled by evaporation of the solvent does not exceed 200° C. for a prolonged period. In general, the exit temperature of the drying gas is adjusted to 50 to 150° C., preferably 100 to 140° C.

The source of the element M can, for example, also be added to a solution of the silver source, molybdenum source and vanadium source which is to be sprayed or to be dried.

The drying generally affords an amorphous product. Appropriately, the product can be compacted and classified into a fraction of suitable particle size, for example between 500-1000 μm.

Typically, a thermal treatment follows, preferably under a controlled atmosphere. Such a thermal treatment is effected statically or preferably movingly under rotating motions of the oven space. Typical temperature regimes for the thermal treatment are in the range from 200 to 800° C., preferably from 250 to 500° C., more preferably from 300 to 400° C. The thermal treatment can take place under inert atmosphere (for example nitrogen or noble gases), oxidizing atmosphere (for example oxygen) or varying atmosphere (first oxidizing, then reducing atmosphere). The person skilled in the art is aware that it is also possible to use mixtures of the gases mentioned. In this context, the term “oxidizing” means that, in the gas stream supplied, after conversion of all oxidizing and reducing agents present, oxidizing agent remains in the gas stream, i.e. an oxidizing gas stream is supplied overall. In this context, the term “reducing” means that, in the gas stream supplied, after conversion of all oxidizing and reducing agents present, reducing agent remains in the gas stream, i.e. a reducing gas stream is supplied overall. In this connection, “inert” means that either no oxidizing agent or reducing agent is supplied, or oxidizing and reducing agents in the gas stream supplied are inert overall, which means that, in the gas stream supplied, after conversion of all oxidizing and reducing agents present, neither oxidizing agent nor reducing agent remains in the gas stream.

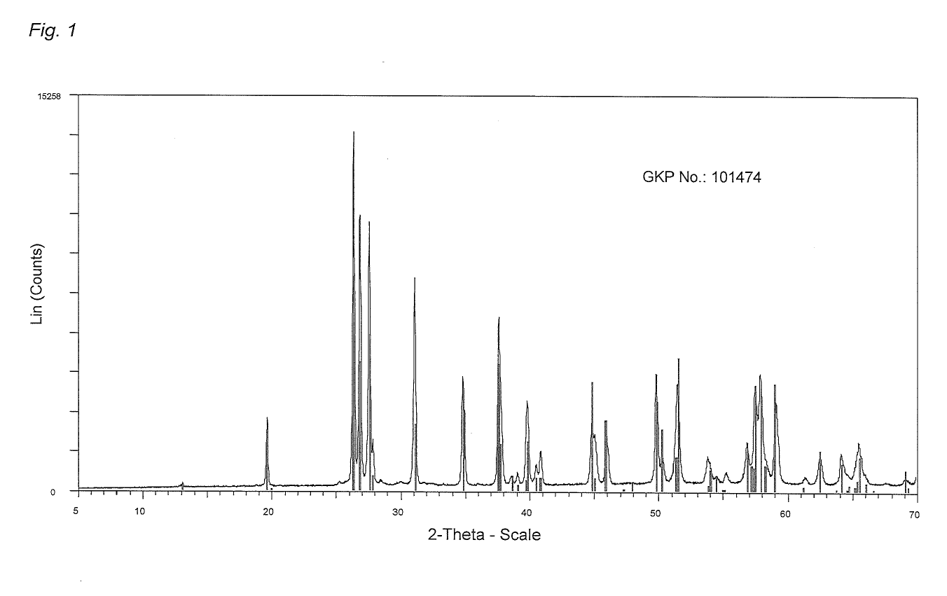

It is possible to effect thermal treatment under standing or flowing atmosphere; preference is given to undertaking treatment under a flowing gas stream, in which case preference is given to a constant fresh gas feed over gas recycling. The composition of the atmosphere can be varied as a function of the calcination temperature and time. Typically, preference is given to a moving thermal treatment, for example by means of rotating calcination drums, agitation or fluidization. For laboratory preparation, preference is given to ovens as in FIG. 1 of DE A 10122027.

The thermal treatment can also be effected under the thermal conditions of the gas phase oxidation in the gas phase oxidation reactor. In this case, a so-called precatalyst is introduced into the reactor and is converted to an inventive catalyst under the thermal conditions of the gas phase oxidation.

Likewise included within the scope of the invention is the washing of the multimetal oxide material (see JP-A 8-57319 or EP-A 1254707) with suitable liquids. Especially aqueous solutions of inorganic or organic acids, and also alcoholic solutions (with and without acids) and aqueous hydrogen peroxide solutions are examples of suitable solvents.

A multimetal oxide in which d is different than 0 can also be obtained by impregnating a multimetal oxide in which d=0 with a source of an element M, for example with a solution of a compound of M, and then drying it.

The multimetal oxide can be used for the partial oxidation in the gas phase in the form of an unsupported catalyst or in the form of a coated catalyst. To this end, the multimetal oxide may be applied to an inert support and/or may be permeated by an inert support.

To modify the mechanical properties, fine, for example nanoscale, oxides, for example TiO₂, SiO₂, ZrO₂, can be added to the multimetal oxide material.

To prepare an unsupported catalyst, the pulverulent multimetal oxide material is compacted to a pressing of the desired catalyst geometry, for example by tableting or extrusion. For unsupported catalyst preparation, as well as the pulverulent mixed metal oxide composition, it is optionally possible to additionally use assistants, for example graphite or stearic acid as lubricants and/or shaping assistants and reinforcing agents such as microfibers of glass, asbestos, silicon carbide or potassium titanate.

To prepare coated catalysts, the pulverulent multimetal oxide material is applied to preshaped inert catalyst supports of suitable geometry.

The inert support materials used may be virtually all prior art support materials as are used advantageously in the preparation of coated catalysts, for example quartz (SiO₂), porcelain, magnesium oxide, tin dioxide, silicon carbide, rutile, alumina (Al₂O₃), aluminum silicate, steatite (magnesium silicate), zirconium silicate, cerium silicate or mixtures of these support materials. Advantageous support materials which should be emphasized are especially steatite and silicon carbide.

The support material is generally nonporous. The expression “nonporous” should be understood in the sense of “nonporous apart from technically ineffective amounts of pores”, since it is technically unavoidable that a small number of pores may be present in the support material which ideally should not comprise any pores.

The shape of the support material is generally not critical for the inventive coated catalysts. For example, catalyst supports in the shape of spheres, rings, tablets, spirals, tubes, extrudates or spall may be used. The dimensions of these catalyst supports correspond to those of catalyst supports typically used to prepare coated catalysts for the gas phase partial oxidation of aromatic hydrocarbons.

To coat the inert support material, known processes are employed. For example, a suspension of the active material or of a precursor can be sprayed onto the catalyst support at elevated temperature in a heated coating drum. Instead of coating drums, it is also possible to use fluidized bed coaters.

The suspension medium is generally water, to which it is preferable to add binders, such as higher alcohols, polyhydric alcohols, e.g. ethylene glycol, 1,4-butanediol or glycerol, dimethylformamide, dimethylacetamide, dimethyl sulfoxide, N-methyl-pyrrolidone or cyclic ureas, such as N,N′-dimethylethyleneurea or N,N′-dimethyl-propyleneurea, or (co)polymers, dissolved or advantageously in the form of an aqueous dispersion, suitable binder contents generally being 10 to 20% by weight, based on the solids content of the suspension. Suitable polymeric binders are, for example, vinyl acetate/vinyl laurate, vinyl acetate/acrylate, styrene/acrylate, vinyl acetate/maleate or vinyl acetate/ethylene copolymers. In the course of a thermal treatment at temperatures of more than 200 to 500° C., the binder escapes as a result of thermal decomposition and/or combustion from the layer applied.

The layer thickness of the catalyst coating or the sum of the layer thicknesses of the coatings which comprise the catalytically active constituents is generally 10 to 250 μm.

The inventive catalysts are used for the partial oxidation of hydrocarbons.

The hydrocarbons may be selected from aliphatic hydrocarbons such as alkanes, especially C₂-C₆-alkanes, cycloalkanes, alkenes, especially C₃-C₆-alkenes, cycloalkenes, alkynes, especially C₃-C₆-alkynes, and cycloalkynes; aromatic hydrocarbons such as benzene or naphthalene, or especially alkylaromatics.

The inventive catalysts are used especially for the partial oxidation of alkylaromatics to aromatic alcohols, aldehydes, carboxylic acids and/or carboxylic anhydrides.

Suitable alkylaromatics are compounds having at least one carbocyclic or heterocyclic aromatic ring structure, which can be converted under the conditions of a gas phase partial oxidation to aldehydes, carboxylic acids and/or carboxylic anhydrides. Suitable alkylaromatic compounds are especially mono- or polyalkylated aromatics, especially methylated and/or ethylated aromatics.

The aromatic parent compounds may bear substituents which behave inertly under the conditions of the partial oxidation, i.e., for example, halogen or the trifluoromethyl, nitro, amino or cyano group. Non-inert substituents are also useful when they are converted to desired substituents under the conditions of the partial oxidation, for example the aminomethyl group or the hydroxymethyl group.

Preferred aromatic hydrocarbons are toluene, o-xylene, m-xylene, p-xylene and methylpyridines.

One embodiment of the process according to the invention relates to the preparation of C8 products of value (o-tolylaldehyde, o-toluic acid, phthalide, phthalic anhydride) from o-xylene.

One embodiment of the process according to the invention relates to the preparation of m-tolylaldehyde from m-xylene.

One embodiment of the process according to the invention for partial oxidation relates to the preparation of phthalic anhydride from o-xylene. For this purpose, the inventive catalysts can be used in combination with other catalysts of different activity, for example prior art catalysts based on vanadium oxide/anatase.

Further embodiments of the process according to the invention for partial oxidation relate to the preparation of benzoic acid and/or benzaldehyde from toluene, or of pyridinecarboxylic acids, such as nicotinic acid, from methylpyridines, such as β-picoline.

The inventive catalysts can be used alone or in combination with other catalysts of different activity, for example prior art catalysts based on vanadium oxide/anatase and/or silver vanadates. The different catalysts are generally arranged in separate catalyst beds, which may be arranged in one or more fixed catalyst beds, in the reactor.

The inventive catalysts are appropriately charged into the reaction tubes of a tubular reactor which are thermostated to the reaction temperature externally, for example by means of a salt melt. The reaction gas is passed over the catalyst bed thus prepared at temperatures of generally 250 to 450° C., preferably 300 to 420° C. and more preferably 320 to 400° C., and at a gauge pressure of generally 0,1 to 2.5 bar, preferably 0.3 to 1.5 bar, with a space velocity of generally 750 to 10 000 h⁻¹, preferably 1500 to 4000 h⁻¹.

The reaction gas supplied to the catalyst is generally obtained by mixing a gas which comprises molecular oxygen and, apart from oxygen, may also comprise suitable reaction moderators and/or diluents, such as steam, carbon dioxide and/or nitrogen, with the alkylaromatic to be oxidized. The gas comprising molecular oxygen comprises generally 1 to 100% by volume, preferably 2 to 50% by volume and more preferably 4 to 30% by volume of oxygen, 0 to 30% by volume, preferably 0 to 20% by volume of steam, and 0 to 50% by volume, preferably 0 to 1% by volume of carbon dioxide, remainder nitrogen. Particularly advantageously, the gas comprising molecular oxygen used is air.

In a preferred embodiment of the process according to the invention, the alkylaromatic is converted over a catalyst whose catalytically active material comprises a multimetal oxide of the formula (I) to an intermediate reaction mixture and the intermediate reaction mixture or fractions thereof is/are converted further over at least one further catalyst.

To this end, the alkylaromatic is, for example, converted first over a bed of the inventive catalyst with partial conversion to a product mixture which may comprise the desired oxidation product, underoxidation products thereof and unconverted alkylaromatic. The product mixture can then be processed further by, alternatively,

a) removing and recycling the unconverted alkylaromatic from the desired oxidation product and the underoxidation products thereof and feeding the stream composed of desired oxidation product and the underoxidation products thereof to one or more further catalyst beds, where the underoxidation products are oxidized selectively to the desired oxidation product; or

b) the product mixture is passed without further workup over a second and optionally further catalyst beds.

Such a reaction regime is found to be particularly advantageous for the preparation of phthalic anhydride from o-xylene. This type of reaction regime achieves a significantly higher phthalic anhydride yield overall than with prior art catalysts alone, since the inventive coated catalysts can oxidize o-xylene significantly more selectively to phthalic anhydride or underoxidation products thereof than is possible in the case of sole use of catalyst systems based on vanadium oxide/anatase according to the prior art.

The invention is illustrated in detail by the appended drawings and the examples which follow.

FIG. 1 shows the x-ray diffractogram of the powder from example 5B.

All x-ray diffractograms were recorded with a diffractometer from the manufacturer Bruker AXS GmbH, 76187 Karlsruhe, instrument designation: D8 Discover with GADDS (General Area Detector Diffraction System). To record the diffractograms, Cu-Kα radiation (40 kV, 40 mA) was used.

### EXAMPLE 1

### Uncalcined Coated Catalyst AgMoVOₑ

A Preparation of the Spray Powder

320 g of ammonium metavanadate (V₂O₅ content of 77.3% by weight, ideal composition: NH₄VO₃, from H. C. Starck) were dissolved at 80° C. in 10 l of deionized water in a glass vessel. This formed a clear yellow solution. 480.4 g of ammonium heptamolybdate hydrate with an MoO₃ content of 81.5% by weight (ideal composition: (NH₄)₆Mo₇O₂₄.4 H₂O, from H. C. Starck) were added to this solution with stirring. This formed a red solution A. In a second glass vessel, 462.1 g of AgNO₃ were dissolved in 2.5 l of demineralized water (solution B). Solution B was subsequently added with stirring to solution A. This formed a yellow suspension. A dropping funnel was used to add 750 g of aqueous NH₄OH solution (25%) dropwise. This formed a clear yellow solution. The solution was heated at 80° C. for 30 minutes. Subsequently, the solution was spray-dried (spray tower from Niro Inc., Mobile Minor 2000).

B Preparation of the Uncalcined Coated Catalyst

65 g of the resulting spray powder were applied to 210 g of spherical support bodies with a diameter of 3.5-4.5 mm (support material=steatite from Ceramtec). To this end, the support was initially charged in a coating drum with internal volume 1.5 l. The drum was set to rotate at 32 revolutions per minute. An atomizer nozzle operated with 150 l (STP)/h of compressed air was used to spray approx. 9 g of a mixture of 1.5 g of glycerol and 7.5 g of water onto the support. At the same time, the powder was introduced into the drum by means of a vibrating chute. On completion of the coating, the coated support bodies were dried at 100° C. in a forced-air drying cabinet (from Heraeus) for 5 hours.

C Calcination of the Coated Catalyst in the Reactor and Catalyst Testing

A 950 mm-long integral reactor with an internal width of 16 mm with an internal thermowell (d=3.17 mm) was charged at a reactor temperature of 50° C. with the uncalcined catalyst spheres coated onto steatite up to a bed length of 66 cm. A further 25 cm of uncoated steatite spheres (d=3.5-4.5 mm) were added to the catalyst charge. 100 l (STP)/h of air were passed through the tube from the top downward. The reactor was heated with electrical heating bands from 50 to 200° C. (20° C./h), and kept at 200° C. for 5 hours for controlled glycerol burnoff. Subsequently, the reactor was heated to 450° C. (20° C./h) and the catalyst was calcined under an air atmosphere (100 l (STP)/h) at 450° C. for 3 h. After the thermal pretreatment, the tube was cooled to 330° C., and 183 l (STP)/h of air and 55.6 l (STP)/h of nitrogen were passed through from the top downward at a loading of 48.2 g of o-xylene/m³ (STP) of gas (1.0% by volume of o-xylene). At 15.0% by volume of oxygen and 4.9% by volume of H₂O, a C8 product of value selectivity of 83.3% was achieved at an o-xylene conversion of 38%. The COₓ selectivity was 13.7% (the COₓ selectivity corresponds to the proportion of the o-xylene converted to combustion products (CO/CO₂); the residual selectivity to 100% corresponds to the proportion of the o-xylene converted to the phthalic anhydride product of value, and to the o-tolylaldehyde, o-toluic acid and phthalide intermediates, and the maleic anhydride, citraconic anhydride and benzoic acid by-products).

A powder x-ray diffractogram was measured on the active material of a deinstalled sample of the catalyst. The active material of the deinstalled sample of the catalyst comprises essentially a mixture of AgMoVO₆ and Ag.

### Example 2

### P-doped Uncalcined Coated Catalyst AgMoVP_{0.006}Oₑ

A Preparation of the Spray Powder

The spray powder was prepared analogously to example 1A.

B Preparation of the Uncalcined Coated Catalyst

65 g of the resulting spray powder were applied to 210 g of spherical support bodies with a diameter of 3.5-4.5 mm (support material=steatite from Ceramtec). To this end, the support was initially charged in a coating drum with internal volume 1.5 l. The drum was set to rotate at 32 revolutions per minute. An atomizer nozzle operated with 150 l (STP)/h of compressed air was used to spray approx. 9 g of a mixture of 1.7 g of glycerol, 7.1 g of water and 0.2 g of phosphoric acid onto the support. At the same time, the powder was introduced into the drum by means of a vibrating chute. On completion of the coating, the coated support bodies were dried at 100° C. in a forced-air drying cabinet (from Heraeus) for 5 hours. By means of atomic spectrometry, the molar P/Ag ratio was determined to be 0.006.

C Calcination of the Coated Catalyst in the Reactor and Catalyst Testing 35

A 950 mm-long integral reactor with an internal width of 16 mm with an internal thermowell (d=3.17 mm) was charged at a reactor temperature of 50° C. with the uncalcined catalyst spheres coated onto steatite up to a bed length of 66 cm. A further 25 cm of uncoated steatite spheres (d=3.5-4.5 mm) were added to the catalyst charge. 100 l (STP)/h of air were passed through the tube from the top downward. The reactor was heated with electrical heating bands from 50 to 200° C. (20° C./h), and kept at 200° C. for 5 hours for controlled glycerol burnoff. Subsequently, the reactor was heated to 450° C. (20° C./h) and the catalyst was calcined under an air atmosphere (100 l (STP)/h) at 450° C. for 22.0 h. After cooling to 330° C., 183 l (STP)/h of air and 55.6 l (STP)/h of nitrogen were passed through from the top at a loading of 48.2 g of o-xylene/m³ (STP) of gas (1.0% by volume of o-xylene). At 20.0% by volume of oxygen and 4.9% by volume of H₂O, a C8 product of value selectivity of 82.0% was achieved at an o-xylene conversion of 43.5%. The COₓ selectivity was 15.0% (the COₓ selectivity corresponds to the proportion of the o-xylene converted to combustion products (CO/CO₂); the residual selectivity to 100% corresponds to the proportion of the o-xylene converted to the phthalic anhydride product of value, and to the o-tolylaldehyde, o-toluic acid and phthalide intermediates, and the maleic anhydride, citraconic anhydride and benzoic acid by-products).

### Example 3

### P-doped Uncalcined Coated Catalyst AgMo_{0.9}VW_{0.1}P_{0.007}Oₑ

A Preparation of the Spray Powder

160 g of ammonium metavanadate (V₂O₅ content of 77.3% by weight, ideal composition: NH₄VO₃, from H. C. Starck) were dissolved at 80° C. in 5 l of deionized water in a glass vessel. This formed a clear yellow solution. 208.2 g of ammonium heptamolybdate hydrate with an MoO₃ content of 81.5% by weight (ideal composition: (NH₄)₆Mo₇O₂₄.4 H₂O, from H. C. Starck) were added to this solution with stirring. This formed a red solution. 45 g of ammonium metatungstate with a tungsten content of 73.5% by weight (ideal composition: (NH₄)H₂W₁₂O₄₀.H₂O, from H. C. Starck) were added with stirring to the red solution. This formed a red solution A. In a second glass vessel, 231 g of AgNO₃ were dissolved in 1.25 l of demineralized water (solution B). Solution B was subsequently added with stirring to solution A. This formed a yellow suspension. A dropping funnel was used to add 375 g of aqueous NH₄OH solution (25%) dropwise. This formed a clear yellow solution. The solution was heated at 80° C. for 30 minutes. Subsequently, the solution was spray-dried (spray tower from Niro Inc., Mobile Minor 2000).

B Preparation of the Uncalcined Coated Catalyst

65 g of the resulting spray powder were applied to 210 g of spherical support bodies with a diameter of 3.5-4.5 mm (support material=steatite from Ceramrec). To this end, the support was initially charged in a coating drum with internal volume 1.5 l. The drum was set to rotate at 32 revolutions per minute. An atomizer nozzle operated with 150 l (STP)/h of compressed air was used to spray approx. 9 g of a mixture of 1.7 g of glycerol, 7.1 g of water and 0.2 g of phosphoric acid onto the support. At the same time, the powder was introduced into the drum by means of a vibrating chute. On completion of the coating, the coated support bodies were dried at 100° C. in a forced-air drying cabinet (from Heraeus) for 5 hours. By means of atomic spectrometry, the molar P/Ag ratio was determined to be 0.007.

C Calcination of the Coated Catalyst in the Reactor and Catalyst Testing

A 950 mm-long integral reactor with an internal width of 16 mm and internal thermowell (d=3.17 mm) was charged at 50° C. with the uncalcined catalyst KD 380 (coated steatite spheres) up to a bed length of 66 cm. A further 25 cm of uncoated steatite spheres (d=3.5-4.5 mm) were added to the catalyst charge. The metal tube was electrically heated with heating bands. 100 l (STP)/h of air were passed through the tube from the top downward. First, the reactor was heated to 200° C. in 20° C./h steps and the glycerol was burnt off in a controlled manner for 5 h. Subsequently, the reactor was heated to 450° C. (20° C./h) and the catalyst was calcined at 450° C. for 22 h. After this thermal pretreatment, the reactor was cooled to 330° C. and the catalyst was laden with 0.5% by volume of o-xylene at 15% by volume of oxygen and 4.9% by volume of H₂O. At 119 l (STP)/h of air, and a loading of 47 g/m³ (STP) and an o-xylene conversion of 37%, a C8 product of value selectivity of 84.2% was achieved. The COₓ selectivity was approx. 12% (the COₓ selectivity corresponds to the proportion of the o-xylene converted to combustion products (CO, CO₂); the residual selectivity to 100% corresponds to the proportion of the o-xylene converted to the phthalic anhydride product of value, and to the o-tolylaldehyde, o-toluic acid and phthalide intermediates, and the maleic anhydride, citraconic anhydride and benzoic acid by-products).

### Example 4

### Calcined tablets AgMoVOₑ

A Preparation of the Spray Powder

The spray powder was prepared analogously to example 1A.

B Preparation of Tablets from the Spray Powder

The resulting spray powder was admixed with 3% by weight of graphite and mixed thoroughly in a drum hoop. Subsequently, the mixture was processed in a compacter to give 3 mm×3 mm tablets.

C Preparation of Calcined Tablets

Tablets from example 4B were calcined in a forced-air oven (from Heraeus) at 450° C. for 2 hours under air (300 l (STP)/h).

A portion of tablets were ground, and a powder x-ray diffractogram of the resulting powder was recorded. The powder has essentially a pure AgMoVO₆ phase.

D Catalyst Testing of Tablets

A 950 mm-long integral reactor with an internal width of 16 mm and internal thermowell (d=3.17 mm) was charged with 70 g of tableted catalyst (3.0×3.0 mm pellets) diluted with 129.4 g of steatite spheres (d=3.5-4.5 mm) up to a bed length of 66 cm. A further 25 cm of uncoated steatite spheres (d=3.5-4.5 mm) were added to the catalyst charge. The metal tube was electrically heated with heating bands. 358 l (STP)/h of air loaded with 48 g o-Xylol/m³ (STP) of gas (1.0% by vol. o-Xylol) were passed through the tube from the top downward. At 15.0% by volume of oxygen, 5.2% by volume of H₂O and at a temperature of 390° C., a C8 product of value selectivity of 69.4% was achieved at an o-xylene conversion of 42.7%. The COₓ selectivity was about 24.0% (the COₓ selectivity corresponds to the proportion of the o-xylene converted to combustion products (CO, CO₂); the residual selectivity to 100% corresponds to the proportion of the o-xylene converted to the phthalic anhydride product of value, and to the o-tolylaldehyde, o-toluic acid and phthalide intermediates, and the maleic anhydride, citraconic anhydride and benzoic acid by-products).

### Example 5

### Calcined Coated Catalyst AgMoVOₑ

A Preparation of the Spray Powder

117.6 g of ammonium metavanadate (V₂O₅ content of 77.3% by weight, ideal composition: NH₄VO₃, from H. C. Starck) were dissolved at 80° C. in 6 l of deionized water in a glass vessel. This formed a yellow solution. 176.6 g of ammonium heptamolybdate hydrate with an MoO₃ content of 81.5% by weight (ideal composition: (NH₄)₆Mo₇O₂₄.4 H₂O, from H. C. Starck) were added to this solution with stirring. This formed a red solution A. In a second glass vessel, 169.9 g of AgNO₃ were dissolved in 0.5 l of demineralized water (solution B). Solution B was subsequently added with stirring to solution A. This formed a yellow suspension. The suspension was heated at 80° C. for 30 minutes. Subsequently, the suspension was spray-dried (spray tower from Niro Inc., Mobile Minor 2000).

B Preparation of Calcined Powder

The resulting spray powder was calcined under air in a rotating bulb furnace at 300° C. for 4 hours and then at 500° C. for 2 hours.

A powder x-ray diffractogram of the resulting powder was recorded. From the powder x-ray diffractogram, the following interplanar spacings d [Å±0.04] with the corresponding relative intensities I₁ₑ, [%] were determined: 6.80 (2), 4.53 (20), 3.38 (100), 3.32 (77), 3.24 (75), 3.20 (13), 2.88 (59), 2.57 (32), 2.39 (48), 2.33 (4), 2.30 (5), 2.26 (25), 2.23 (7), 2.21 (11), 2.02 (19), 2.01 (16), 1.97 (15), 1.83 (33), 1.81 (10), 1.77 (30), 1.70 (10), 1.68 (4), 1.66 (5), 1.62 (14), 1.60 (30), 1.59 (33), 1.58 (9), 1.56 (25), 1.51 (4), 1.48 (10), 1.45 (11), 1.42 (14), 1.35 (5.1). It has essentially a pure AgMoVO₆ phase.

The calcined powder was subsequently ground with stainless steel balls (Ø=40 mm) using a vibrating plate through a 100 μm stainless steel screen.

C Preparation of Precalcined Coated Catalyst

52.5 g of the precalcined powder from example 5B were applied to 210 g of spherical support bodies with a diameter of 3.5-4.5 mm (support material=steatite from Ceramtec). To this end, the support was initially charged in a coating drum of internal volume 1.5 l. The drum was set to rotate at 32 revolutions per minute. An atomizer nozzle operated with 150 l (STP)/h of compressed air was used to spray approx. 12 g of a mixture of 2.3 g of glycerol and 9.7 g of water onto the support. At the same time, the powder was introduced into the drum via a vibrating chute. On completion of the coating, the coated support bodies were dried at 250° C. in a forced-air drying cabinet (from Heraeus) for 2.5 hours.

A powder x-ray diffractogram of the coated catalyst obtained was recorded. From the powder x-ray diffractogram, the following interplanar spacings d [Å±0.04] with the corresponding relative intensities Iᵣₑₗ, [%] were determined: 6.78 (9), 4.52 (28), 3.38 (100), 3.32 (81), 3.23 (87), 3.20 (13), 2.88 (65), 2.57 (34), 2.39 (59), 2.33 (6), 2.30 (7), 2.26 (33), 2.22 (11), 2.21 (14), 2.02 (27), 2.01 (20), 1.97 (21), 1.83 (47), 1.81 (15), 1.77 (45), 1.70 (14), 1.68 (6), 1.66 (6), 1.62 (21), 1.60 (48), 1.59 (53), 1.58 (14), 1.56 (40), 1.51 (6), 1.48 (15), 1.45 (18), 1.42 (23), 1.35 (6). It has essentially a pure AgMoVO₆ phase. In addition, it has weak intensities (<5%) of AgO (index card 01-084-1547 of the ICDD PDF-2 index (2006 release)).

D Catalyst Testing

A 950 mm-long integral reactor with an internal width of 16 mm and internal thermowell (d=3.17 mm) was charged with the catalyst prepared (coated steatite spheres) up to a bed length of 66 cm. A further 25 cm of uncoated steatite spheres (d=3.5-4.5 mm) were added to the catalyst charge. The metal tube was electrically heated with heating bands. 122 l (STP)/h of air and 117 l (STP)/h of nitrogen were passed through the tube from the top downward with a loading of 52 g of o-xylene/m³ (STP) of gas (1.0% by volume of o-xylene). At 10.0% by volume of oxygen and 4.9% by volume of H₂O, a C8 product of value selectivity of 84% was achieved at 410° C. at an o-xylene conversion of 22.5%. The COₓ selectivity was 12.3% (the COₓ selectivity corresponds to the proportion of the o-xylene converted to combustion products (CO, CO₂); the residual selectivity to 100% corresponds to the proportion of the o-xylene converted to the phthalic anhydride product of value, and the o-tolylaldehyde, o-toluic acid and phthalide intermediates, and the maleic anhydride, citraconic anhydride and benzoic acid by- products).

A powder x-ray diffractogram was measured on the active material of a deinstalled sample of the catalyst, which detects the following interplanar spacings d [Å±0.04] with the corresponding relative intensities Iᵣₑₗ, [%]: 6.06 (17), 4.53 (14), 4.05 (25), 3.55(29), 3.39 (53), 3.32 (57), 3.24 (42), 3.03 (17), 2.88 (31), 2.73 (17), 2.67 (16), 2.57 (18), 2.39 (29), 2.36 (100), 2.26 (18), 2.04 (32), 2.02 (24), 1.83 (23), 1.81 (11), 1.77 (21), 1.60 (21), 1.59 (24), 1.56 (17), 1.48 (11), 1.44 (39), 1.42 (14). The active material of the deinstalled sample of the catalyst comprises essentially a mixture of AgMoVO₆, Ag (index card 03-065-2671 of the ICDD PDF-2 index (2006 release)) and V_{0.95}Mo_{0.97}O₅ (index card 01-077-0649 of the ICDD PDF-2 index (2006 release)).

### Example 6

### Calcined Coated Catalyst AgMoVOₑ

A Preparation of the Spray Powder

117.64 g of ammonium metavanadate (V₂O₅ content of 77.3% by weight, ideal composition: NH₄VO₃, from H. C. Starck) were dissolved at 80° C. in 6 l of deionized water in a glass vessel. This formed a clear yellow solution. 176.6 g of ammonium heptamolybdate hydrate with an MoO₃ content of 81.5% by weight (ideal composition: (NH₄)₆Mo₇O₂₄.4 H₂O, from H. C. Starck) were added to this solution with stirring. This formed a red solution A. In a second glass vessel, 169.9 g of AgNO₃ were dissolved in 0.5 l of demineralized water (solution B). Solution B was subsequently added with stirring to solution A. This formed an ochre suspension and the temperature fell to 76° C. The suspension was heated to 80° C. and heated at 80° C. for 30 minutes. Subsequently, the suspension was spray-dried (spray tower from Niro Inc., Mobile Minor 2000).

B Preparation of Calcined Powder

The resulting spray powder was orange. The powder was calcined under air in a rotating bulb furnace at 300° C. for 4 hours.

A powder x-ray diffractogram of the resulting powder was recorded. From the powder x-ray diffractogram, the following interplanar spacings d [Å±0.04] were detected with the corresponding relative intensities Iᵣₑₗ, [%]: 6.77 (4.3), 4.53 (19.1), 3.39 (100), 3.32 (83), 3.23 (82.2), 2.88 (55.5), 2.57 (34), 2.39 (50.6), 2.33 (5.9), 2.31 (5.8), 2.26 (29), 2.23 (9), 2.21 (12.5), 2.02 (19.3), 2.01 (16.4), 1.97 (16.3), 1.83 (30.4), 1.81 (12.5), 1.77 (31.7), 1.70 (12.1), 1.66 (7.3), 1.62 (15.4), 1.60 (33.5), 1.59 (29.7), 1.56 (28.1), 1.51 (6.2), 1.48 (11.4), 1.45 (10.6), 1.42 (16.8), 1.35 (5.1). It has essentially an AgMoVO₆ phase.

C Preparation of Precalcined Coated Catalyst

23.3 g of the resulting powder were applied to 210 g of spherical support bodies with a diameter of 3.2-4.5 mm (support material=steatite from Ceramtec). To this end, the support was initially charged in a coating drum of internal volume 1.5 l. The drum was set to rotate at 32 revolutions per minute. An atomizer nozzle operated with 150 l (STP)/h of compressed air was used to spray approx. 12 ml of a mixture of glycerol and water (glycerol: water weight ratio=19.3: 100) onto the support. At the same time, the powder was introduced into the drum via a vibrating chute.

On completion of the coating, the coated support bodies were dried at 100° C. in a forced-air drying cabinet (from Heraeus) over 5 hours, and then aftertreated thermally in a muffle furnace at 500° C. over 2 hours. The weight of the catalytically active material thus applied was 9.6% by weight based on the total weight of the finished catalyst.

A powder x-ray diffractogram of the resulting active material was recorded (FIG. 1). From the powder x-ray diffractogram, the following interplanar spacings d [Å±0.04] were detected with the corresponding relative intensities Iᵣₑₗ [%]: 4.53 (26.6), 3.38 (100), 3.32 (91.6), 3.24 (94.2), 2.88 (70.2), 2.57 (43.1), 2.39 (46), 2.33 (6.7), 2.30 (6.6), 2.26 (44.6), 2.22 (9.5), 2.21 (12.9), 2.02 (27.1), 1.97 (18.7), 1.83 (34.6), 1.81 (17.1), 1.77 (49.8), 1.70 (13), 1.68 (5.8), 1.66 (7.4), 1.62 (19.8), 1.60 (37.1), 1.59 (48.4), 1.56 (30.5), 1.48 (13.2), 1.45 (14.1), 1.42 (20.8), 1.36 (6.2). The active material of the catalyst has a pure AgMoVO₆ phase.

D Catalyst Testing

A 950 mm-long integral reactor with an internal width of 16 mm and internal thermowell (d=3.17 mm) was charged with the catalyst prepared (coated steatite spheres) up to a bed length of 66 cm. A further 25 cm of uncoated steatite spheres (d=3.5-4.5 mm) were added to the catalyst charge. The metal tube was electrically heated with heating bands. 122 l (STP)/h of air and 117 l (STP)/h of nitrogen were passed through the tube from the top downward with a loading of 52 g of o-xylene/m³ (STP) of gas (1.0% by volume of o-xylene). At 10.0% by volume of oxygen and 4.9% by volume of H₂O, a C8 product of value selectivity of 83.4% was achieved at 430° C. at an o-xylene conversion of 9.4%. The COₓ selectivity was 14.3% (the COₓ selectivity corresponds to the proportion of the o-xylene converted to combustion products (CO, CO₂); the residual selectivity to 100% corresponds to the proportion of the o-xylene converted to the phthalic anhydride product of value, and to the o-tolylaldehyde, o-toluic acid and phthalide intermediates, and the maleic anhydride, citraconic anhydride and benzoic acid by-products).

A powder x-ray diffractogram was measured on the active material of a deinstalled sample of the catalyst, which had the following interplanar spacings d [Å] with the corresponding relative intensities I_{ref}, [%]: 4.53 (17.1), 3.38 (89.8), 3.32 (86.2), 3.23 (100), 2.88 (73.4), 2.57 (46.2), 2.39 (63.2), 2.33 (8.1), 2.30 (7.5), 2.26 (52.4), 2.22 (9.9), 2.21 (13.7), 2.02 (28.3), 1.97 (20.8), 1.83 (36.9), 1.81 (16.3), 1.77 (46.1), 1.70 (12.4), 1.68 (4.4), 1.66 (6.9), 1.62 (19.5), 1.60 (33.4), 1.59 (40.9), 1.56 (30.6), 1.48 (11.1), 1.45 (13.2), 1.42 (18.5), 1.36 (5.2). The active material of the deinstalled sample of the catalyst has essentially an AgMoVO₆ phase.

### Example 7

### Preparation and Testing of the Catalyst Spall of AgMoVOₑ

The calcined powder from example 6B was compacted by means of a compactor (from Paul-Otto Weber GmbH) and classified to a fraction between 500 and 1000 μm. Catalyst spall was initially charged in porcelain dishes on a shaker and impregnated with different aqueous metal salt solutions (H₃BO₃, LiNO₃, H₃PO₄, Cu(NO₃)₂, Fe(NO₃)₃, Sb(CH₃COO)₃, Ce(NO₃)₃, (NH₄)NbO(C₂O₄)₂.xH₂O, Bi(NO₃)₃, (NH₄)₆H₂W₁₂O₄₀.x H₂O) in different loadings, dried under air on the shaker for 30 min and then dried further in a drying cabinet for 18 h. The dry active materials were screened to a fraction between 500-1000 μm and tested in a reactor.

The catalytic testing was effected on 1 ml of the sample in a 48-tube test reactor according to DE 198 09 477.9. The catalysts were tested at an o-xylene concentration between 1-3% by volume, an oxygen concentration between 7-17% by volume, a water concentration between 5-10% by volume, a GHSV between 1000-10 000 h⁻¹ within a temperature range between 280-350° C.

Tables 2-4 show an extract of the active materials tested and the results obtained with regard to CO₂ for the doped and undoped active materials.

TABLE 1
Results of the catalyst testing in a single-tube reactor

| | | | | O₂ | | | |
|---|---|---|---|---|---|---|---|
| | | Temper- | | conc. | C(o- | | |
| Example | | ature | GHSV | [% by | xylene) | S(C8) | S(COₓ) |
| No. | Catalyst | (° C.) | (h⁻¹) | vol.] | [%] | [%] | [%] |
| 1 | Coated catalyst | 410 | 2000 | 15 | 38.1 | 83.3 | 13.7 |
| | AgMoVOₑ | | | | | | |
| 2 | Coated catalyst | 410 | 995 | 20 | 43.5 | 82.0 | 15.0 |
| | AgMoVP_{d}Oₑ | | | | | | |
| 3 | Coated catalyst | 410 | 995 | 15 | 36.9 | 84.2 | 11.9 |
| | AgMo_{0.9}VW_{0.1}P_{d}Oₑ | | | | | | |
| 4 | AgMoVOₑ | 390 | 2994 | 15 | 42.7 | 69.4 | 24.0 |
| | tablets | | | | | | |
| 5 | Coated catalyst | 410 | 1997 | 10 | 22.5 | 84.1 | 12.3 |
| | AgMoVOₑ | | | | | | |
| 6 | Coated catalyst | 430 | 1997 | 10 | 9.4 | 83.4 | 14.3 |
| | AgMoVOₑ | | | | | | |

TABLE 2
Results of the catalyst testing in a 48-tube reactor (example 7; undoped
and doped AgMoVOₑ) at 1% by volume of o-xylene, 11% by volume of
oxygen, 5% by volume of water, GHSV = 10 000 h⁻¹, 350° C.

| Doping metal (loading) | C(o-xylene) [%] | S(CO₂) [%] |
|---|---|---|
| Undoped AgMoVO₆ | 18.54 | 12.20 |
| P (0.1% by weight) | 44.61 | 10.94 |
| Ce (0.1% by weight) | 14.18 | 12.00 |
| Ce (1% by weight) | 23.89 | 10.41 |
| Sb (0.1% by weight) | 16.76 | 10.63 |
| Sb (1% by weight) | 17.70 | 10.21 |
| Bi (0.1% by weight) | 18.58 | 11.77 |
| Bi (1% by weight) | 16.28 | 10.78 |
| Cs (0.1% by weight) | 9.13 | 10.53 |
| Nb (0.1% by weight) | 17.85 | 10.64 |
| Nb (1% by weight) | 20.85 | 10.59 |
| W (0.1% by weight) | 19.09 | 11.68 |
| W (1% by weight) | 24.81 | 10.46 |
| B (0.1% by weight) | 22.44 | 11.88 |
| B (1% by weight) | 18.06 | 11.43 |

TABLE 3
Results of the catalyst testing in a 48-tube reactor (example 7; undoped
and doped AgMoVOₑ) at 3% by volume of o-xylene, 17% by volume of
oxygen, 5% by volume of water, GHSV = 6500 h⁻¹, T = 330° C.

| Doping metal (loading) | C(o-xylene) [%] | S(CO₂) [%] |
|---|---|---|
| Undoped AgMoVO₆ | 15.53 | 12.31 |
| Ce (0.1% by weight) | 14.71 | 11.40 |
| Ce (1% by weight) | 28.24 | 12.20 |
| Sb (0.1% by weight) | 14.18 | 11.31 |
| Sb (1% by weight) | 14.50 | 10.47 |
| Bi (0.1% by weight) | 15.85 | 11.69 |
| Bi (1% by weight) | 18.12 | 11.64 |
| Cs (0.1% by weight) | 12.93 | 8.09 |
| Nb (0.1% by weight) | 15.37 | 11.72 |
| Nb (1% by weight) | 15.11 | 11.58 |
| W (0.1% by weight) | 16.38 | 11.91 |
| B (0.1% by weight) | 23.73 | 10.98 |
| B (1% by weight) | 15.62 | 11.85 |
| Cu (0.1% by weight) | 16.77 | 11.57 |
| Fe (0.1% by weight) | 24.36 | 11.62 |
| Fe (1% by weight) | 14.11 | 11.56 |
| Li (0.1% by weight) | 18.19 | 12.94 |

TABLE 4
Results of the catalyst testing in a 48-tube reactor (example 7; undoped
and doped AgMoVOₑ) at 1% by volume of o-xylene, 7% by volume of
oxygen, 5% by volume of water, GHSV = 2000 h⁻¹, T = 290° C.

| Doping metal (loading) | C(o-xylene) [%] | S(CO₂) [%] |
|---|---|---|
| Undoped AgMoVO₆ | 18.75 | 19.35 |
| P (0.1% by weight) | 31.46 | 10.71 |
| P (1% by weight) | 23.78 | 8.00 |
| Ce (0.1% by weight) | 15.06 | 14.23 |
| Ce (1% by weight) | 36.21 | 10.52 |
| Sb (0.1% by weight) | 9.19 | 17.04 |
| Sb (1% by weight) | 23.76 | 7.78 |
| Bi (0.1% by weight) | 20.23 | 9.88 |
| Bi (1% by weight) | 16.41 | 10.31 |
| Cs (0.1% by weight) | 22.90 | 17.74 |
| Nb (0.1% by weight) | 16.52 | 9.67 |
| Nb (1% by weight) | 17.43 | 9.71 |
| W (0.1% by weight) | 12.64 | 12.64 |
| W (1% by weight) | 10.96 | 14.45 |
| B (0.1% by weight) | 18.79 | 11.02 |
| B (1% by weight) | 30.78 | 8.80 |
| Cu (0.1% by weight) | 17.94 | 9.09 |
| Cu (1% by weight) | 16.40 | 13.05 |
| Fe (0.1% by weight) | 20.21 | 13.03 |
| Fe (1% by weight) | 33.84 | 8.87 |

1-13. (canceled)

14. A catalyst for the partial oxidation of hydrocarbons in the gas phase, comprising a multimetal oxide of the general formula (I)
AgₐMo_{b}V_{c}M_{d}Oₑ*f H₂O (I)
in which
M is at least one element selected from the group consisting of Li, Na, K, Rb, Cs, Be, Mg, Ca, Sr, Ba, B, Al, Ga, In, Si, Sn, Pb, P, Sb, Bi, Y, Ti, Zr, Hf, V, Nb, Ta, Cr, W, Mn, Re, Fe, Ru, Os, Co, Rh, Ir, Ni, Pd, Pt, Cu, Au, Zn, Cd, La, Ce, Pr, Nd, Sm, Eu, Gd, Tb, Dy, Ho, Er, Tm, Yb and Lu, U,
a has a value of 0.5 to 1.5,
b has a value of 0.5 to 1.5,
c has a value of 0.5 to 1.5,
a+b+c has the value of 3,
d has a value of less than 1,
e is a number which is determined by the valency and frequency of the elements in the formula I other than oxygen,
f has a value of 0 to 20,
which is present in a crystal structure whose powder x-ray diffractogram is characterized by reflections at at least 5 interplanar spacings selected from d=4.53, 3.38, 3.32, 3.23, 2.88, 2.57, 2.39, 2.26, 1.83, 1.77 Å (±0.04 Å).

15. The catalyst according to claim 14, wherein
a has a value of 0.8 to 1.2.

16. The catalyst according to claim 14, wherein
b has a value of 0.8 to 1.2.

17. The catalyst according to claim 14, wherein
c has a value of 0.8 to 1.2.

18. The catalyst according to claim 14, wherein
M is at least one element selected from the group consisting of P, Ce, Sb, Bi, Cs, Nb, W, B, Cu and Fe.

19. The catalyst according to claim 14, wherein
d has the value 0.

20. The catalyst according to claim 14, wherein the multimetal oxide has been applied to an inert support and/or is permeated by an inert support.

21. The catalyst according to claim 15, wherein
b has a value of 0.8 to 1.2,
c has a value of 0.8 to 1.2,
M is at least one element selected from the group consisting of P, Ce, Sb, Bi, Cs, Nb, W, B, Cu and Fe, and
d has the value 0.

22. A process for partial oxidation of hydrocarbons, in which a gaseous stream comprising passing at least one hydrocarbon and molecular oxygen over a bed of the catalyst according to claim 14.

23. The process according to claim 22, wherein the hydrocarbon is an alkylaromatic.

24. The process according to claim 22, wherein the alkylaromatic is selected from the group consisting of toluene, o-xylene, m-xylene and p-xylene.

25. The process according to claim 22, wherein the hydrocarbon is converted over a catalyst whose catalytically active material comprises a multimetal oxide of the formula Ito an intermediate reaction mixture, and the intermediate reaction mixture or fractions thereof is converted further over at least one further catalyst.

26. The process according to claim 25, wherein the catalytically active material of the further catalyst comprises titanium dioxide, vanadium pentoxide or silver vanadate.

27. A process for preparing a multimetal oxide of the general formula (I)
AgₐMo_{b}V_{c}M_{d}Oₑ.f H₂O (I)
in which
M is at least one element selected from the group consisting of Li, Na, K, Rb, Cs, Be, Mg, Ca, Sr, Ba, B, Al, Ga, In, Si, Sn, Pb, P, Sb, Bi, Y, Ti, Zr, Hf, V, Nb, Ta, Cr, W, Mn, Re, Fe, Ru, Os, Co, Rh, Ir, Ni, Pd, Pt, Cu, Au, Zn, Cd, La, Ce, Pr, Nd, Sm, Eu, Gd, Tb, Dy, Ho, Er, Tm, Yb and Lu, U,
a has a value of 0.5 to 1.5,
b has a value of 0.5 to 1.5,
c has a value of 0.5 to 1.5,
a+b+c has the value of 3,
d has a value of less than 1,
e is a number which is determined by the valency and frequency of the elements in the formula I other than oxygen,
f has a value of 0 to 20,
which comprises mixing at least one silver source, at least one molybdenum source, at least one vanadium source and optionally a source of the element M in wet form, the mixture is dried and the solid obtained is treated thermally.
